(19) United States
(12) Patent Application Publication
(10) Pub. No.: US 2014/0081122 A1
(43) Pub. Date: Mar. 20, 2014

(54) METHOD FOR GENERATING A PET IMAGE DATA RECORD OF A MOVED EXAMINATION OBJECT AND FACILITY THEREFOR

(71) Applicant: SIEMENS AKTIENGESELLSCHAFT, Munich (DE)

(72) Inventor: Sebastian SCHMIDT, Weisendorf (DE)

(73) Assignee: SIEMENS AKTIENGESELLSCHAFT, Munich (DE)

(21) Appl. No.: 14/022,412

(22) Filed: Sep. 10, 2013

(30) Foreign Application Priority Data
Sep. 19, 2012 (DE) 102012216759.5

(51) Int. Cl. A61B 5/00

(52) U.S. Cl. CPC A61B 5/0035
USPC 600/411; 600/425; 600/427

(57) ABSTRACT

A method is disclosed for generating a PET image data record or a SPECT image data record of an at least partially moved examination object. The method includes recording or calculation of at least one first PET start image data record or one first SPECT start image data record; recording of at least one first anatomy image data record with an imaging facility imaging anatomical features; recording of at least one second anatomy image data record with the imaging facility imaging the anatomical features; determination of at least one transformation requirement from the first and second anatomy image data record; and generation of at least one PET image data record or one SPECT image data record by the application of the at least one transformation requirement onto the PET start image data record or the SPECT start image data record respectively. An embodiment further relates to an imaging facility.

### PRIORITY STATEMENT

The present application hereby claims priority under 35 U.S.C. §119 to German patent application numbers DE 102012216759.5 filed Sep. 19, 2012, the entire contents of which are hereby incorporated herein by reference.

### FIELD

At least one embodiment of the present invention generally relates to a method for generating a PET image data record of an at least partially moved examination object.

### BACKGROUND

Positron-emission-tomography, abbreviated to PET, is an imaging method with which the distribution of a radioactive substance in an examination object can be represented. With PET, radionuclides emitting positrons are used, wherein, for the recording of measurement data, a detector ring is arranged around the examination object. At the annihilation of an emitted positron with an electron, two photons are released, which move apart from one another in opposite directions. If two photons are acquired by the detector ring within a specified period of time, this is evaluated as coincidence and therefore as an annihilation event. Because the photons, starting from the departure point, have moved apart from one another, the line connecting the detectors is referred to as the “line of response”, abbreviated to LOR.

One individual detected annihilation event therefore still does not provide space-resolved information. It is only by the detection of a plurality of annihilation events and the superimposing of a plurality of lines detected in this way that a PET image data record can be generated.

In this situation, a connection pertains between the radioactivity of the radionuclide used and the temporal duration for the creation of a PET image data record. In order to keep the radiation burden low on the patient, as an examination object, weakly radioactive substances are used. As a result, the measuring time for the recording of a PET image data record is about one minute. PET image data records therefore cannot be represented in “real time”. The term “real-time imaging” is understood to mean the representation of movements, wherein the type of movement determines the time slot, and several image data records are to be recorded in this time slot.

In this situation, a distinction is to be made between actual real-time imaging and pseudo-real-time imaging. With actual real-time imaging, several imaging data records are recorded in the specified time slot, and with pseudo real-time imaging, only parts of the image data records. An example of pseudo-real-time imaging is high-resolution magnetic resonance tomography (MRT) of the heart of a mouse. With a heart rate of 600 l/min, it is not possible with the present state of the art for, for example, five or more complete flash image data records to be recorded in one single cycle of the periodic heart movement. Accordingly, the heart movement is acquired with an EKG, as a function of a specific section of the EKG signal, for example the R-wave, a trigger signal is generated, and one or more k-space lines are recorded per flash image data record.

The recording of a complete unit of image data records therefore lasts several minutes. Nevertheless, after the post-processing steps of the measurement data, which are always necessary, information is available with regard to the entire movement of the heart of the mouse, wherein the phases of the heart beat are represented with a plurality of MR image data records.

With actual real-time imaging, several image data records are acquired in the specified time slot. For example, the movement of the internal organs of an examination object is represented by means of TrueFisp imaging within the framework of a magnetic resonance examination. Other rapid imaging techniques of this imaging modality are EPI, FLASH, HASTE and PROPELLER. In this situation, spin echo-based methods are preferably used with tissues with air inclusions, and gradient echo-based methods with homogeneous tissues.

From DE 102 31 061 A1 a method is known with which it is intended that the movement artifacts in the PET image data records are to be reduced. In this situation, the measurement data of a PET image data record is movement-corrected on the basis of several magnetic resonance or computed tomography image data records. A real-time representation of PET image data records is not disclosed, however.

### SUMMARY

A method and facility are provided which makes possible a determination and representation of a PET image data record or a SPECT image data record with increased time resolution while maintaining a low radiation burden.

A method is disclosed for generating a PET image data record or SPECT image data record. Advantageous further developments of the invention are the subject matter of the dependent claims.

Hereinafter the explanations relate solely to PET image data records. They also apply exactly to SPECT image data records, however.

According to at least one embodiment of the invention, a PET start image data record and an anatomy image data record are first recorded. In this situation, the term “recording of an image data record” is meant the recording, by a detector corresponding to one of the forms of imaging, of signals, also referred to as measurement data or raw data, storing them at least in a volatile memory, appropriate post-processing of the method, and then representing or storing them. Post-processing steps within the framework of magnetic resonance imaging are, for example, the sorting of the measurement data, the Fourier transformation of the measurement data or, respectively, of the sorted measurement data or what is referred to as zero-filling.

In addition to this, the invention also relates to an imaging facility for generating at least one PET or SPECT image data record, comprising an imaging facility which images anatomical features, in particular a magnetic resonance or an X-ray or a computed tomography or an ultrasound facility, a PET facility or a SPECT facility, and a control facility.

### BRIEF DESCRIPTION OF THE DRAWINGS

Further advantages, features, and details of the present invention are derived from the following description of advantageous embodiments of the invention.

The figures show

FIG. 1 a flow chart of the method according to an embodiment of the invention,

FIG. 2 the calculation of PET image data records with complete transformation requirements,

FIG. 3 the combined representation of MR and PET image data records

FIG. 4 the calculation of PET image data records with successive transformation requirements,

FIG. 5 the movement correction of PET measurement data,

FIG. 6 the calculation of PET image data records with movement correction

FIG. 7 PET pseudo-real-time image data records,

FIG. 8 a movement check of an examination region,

FIG. 9 the determination of a moved part region in an examination object, and

FIG. 10 the determination of a moved section

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to at least one embodiment of the invention, a PET start image data record and an anatomy image data record are first recorded. In this situation, the term “recording of an image data record” is meant the recording, by a detector corresponding to one of the forms of imaging, of signals, also referred to as measurement data or raw data, storing them at least in a volatile memory, appropriate post-processing of the method, and then representing or storing them. Post-processing steps within the framework of magnetic resonance imaging are, for example, the sorting of the measurement data, the Fourier transformation of the measurement data or, respectively, of the sorted measurement data or what is referred to as zero-filling.

The steps for the recording of the PET start image data record and of the first anatomy image data record are interchangeable in temporal sequence, and in particular can also take place simultaneously.

In this situation, an anatomy image data record is recorded with an appropriate imaging modality, also referred to hereinafter as anatomical imaging modality. This may involve, for example, a magnetic resonance facility or a computed tomography facility or an X-ray facility or an ultrasound facility. A precondition is that the imaging modality allows for a higher time resolution with comparable spatial resolution in comparison with PET imaging. It must at least allow for the determination of transformation requirements.

It is therefore possible, with the anatomical imaging modality, for an improved time resolution, in particular real-time imaging, to be realized. In order to be able to transfer this time resolution to the PET image data records, after the first anatomy image data record a second anatomy image data record is recorded. The first anatomy image data record can in this situation be recorded with any desired method, in particular with low time resolution. It only needs to be known at what point in time a periodic movement was recorded. Alternatively, the first anatomy image data record can be recorded with an unmoved examination object or an unmoved part of the examination object.

Taking the first anatomy image data record as a starting point, via the second anatomy image data record an item of movement information can be determined, and, resulting from that, one or more transformation requirements. In particular, an individual transformation requirement can be determined for every image element, at least in one region of the anatomy image data records. Alternatively, the first or second anatomy image data record can be segmented, and for any desired segment a transformation requirement can be determined. Image elements are usually also referred to as pixels.

The transformation requirement is a two-dimensional or three- or more dimensional numerical vector, which indicates how an image element has moved, starting out from the first anatomy image data record. As the first anatomy image data record, any anatomy image data record can be used which shows the examination object at the point in time or in the position in which the PET start image data record was recorded. A distinction is to be made in this case between periodic and irregular movements. Periodic movements are caused, for example, by the heart beat or the respiration of an examination object. Irregular movements are, among others, movements of the entire examination object, due, for example, to a movement of the couch on which the examination object is being held.

With regard to periodic movements, reference is again made hereinafter to the example of a beating heart. The movement of the heart is subdivided into two main sections, the systole and the diastole. In the systole, the heart muscle contracts and the blood is pumped out of the heart. During the diastole, by contrast, blood flows into the heart.

The duration of the systole is relatively constant, while the duration of the diastole varies with a change in the heart rate. The beginning of the outflow phase correlates in the EKG with the R-wave, and can therefore be used as a marker for the triggering. With a constant heart rate, the diastole and the systole exhibit consistent changes.

Taking as a starting point, for example, the R-wave, or any other marker which indicates a specific point in time of the periodic heart movement, an image data record which images the heart can be determined or measured at any point in time of the heart movement. In particular, it is possible for a first anatomy image data record to be measured or calculated before the beginning of the systole, and a second anatomy image data record at the end of the systole. It is then possible, from the first and second anatomy image data records, for any desired number of transformation requirements to be derived, which contain an item of information indicating how a specific image element or a predetermined region with several image elements has changed. In the case of the beating heart, the creation of transformation requirements is particularly demanding, since both translational and rotational movements as well as changes in the size of the heart are to be taken into account.

Once the transformation requirement has been determined, it is applied to the PET start image data record. In this situation, the PET start image data record and the first anatomy image data record must therefore represent the examination object at the same point in time of a periodic movement, in order for the transformation requirement to be used such that the movement carried out can be transferred to the PET start image data record. In this way, the high time resolution of the imaging facility which is imaging the anatomical features can be transferred to the PET image data records.

In the case of pseudo-real-time imaging, a complete set of anatomy image data records can be recorded. If it is intended that the anatomy image data records should be used in the performance of an interventional procedure, then a first set of anatomy image data records can be recorded before the beginning of the intervention, and during the intervention further records can be recorded. One of the anatomy image data records can be determined as the first anatomy image data record, and the further anatomy image data records of the set are then second anatomy image data records.

The transformation requirements are determined in that, for every second anatomy image data record, starting from the first anatomy image data record, at least one transformation requirement is determined. These transformation requirements are applied to the PET start image data record, and in this way a set of PET image data records is acquired which is a set of pseudo-real-time PET image data records. The determination of transformation requirements, starting from a consistent first anatomy image data record, is referred to hereinafter as a complete calculation of a transformation requirement.

In an alternative embodiment, at least one transformation requirement is acquired, in that a start is made with two anatomy image data records following consecutively in the temporal sequence of the periodic movement, and at least one transformation requirement is determined. This is then repeated with all the anatomy image data records until at least one transformation requirement has been determined for every anatomy image data record and for the following anatomy image data record. Accordingly, every anatomy image data record is at one time a first and at one time a second anatomy image data record. In this case, starting from the first measured PET start image data record, each newly-calculated PET image data record becomes a new PET start image data record. This way of calculating the transformation requirement is referred to hereinafter as successive calculation.

With actual real-time imaging it is also possible, with the recording of several second anatomy image data records, for the transformation requirements to be determined, starting from the first anatomy image data record, completely or successively.

In at least one embodiment, the at least one transformation requirement can take place by way of an elastic registration of the first anatomy image data record with the second anatomy image data record. With elastic image registration methods, complex local distortions can be particularly well determined.

Particularly advantageously, in each case a part section of the first and second anatomy image data record can be used for the determination of the at least one transformation requirement. In this situation it is of course always a precondition that the anatomy image data records image at least the region which represents the examination region of the examination object, and from which the PET signals for the PET imaging derive. Otherwise, the determination of a transformation requirement or the transfer to the PET start image data record would not be possible.

In this situation, for example with magnetic resonance imaging, it is necessary for the entire cross-section of the examination object to be scanned in order to avoid folding in the image creation. Accordingly, a large region is always recorded around the part of the examination object from which the PET signals are expected. The determination of transformation requirements from the anatomy image data records is only necessary, however, for the region in which the PET start image data record exhibits signals. Accordingly, starting from the PET start image data record, what is referred to as a “region of interest” (ROI) can be determined in the first anatomy image data record, for which transformation requirements are to be determined.

In this situation, a threshold value can be specified for the intensity of the signal of the PET start image data record, in order to delimit the part region.

In at least one embodiment, the part region can be automatically determined from the first and/or second anatomy image data record. If it is intended that a specific organ is to be examined, it is possible, by inputting it by means of an input facility, and using pattern recognition algorithms, for this organ to be automatically separated in an anatomy image data record. This information also allows for the use of optimized elastic registration methods, which, before the determination of the transformation requirements for each organ of an examination object, can be started, for example, with an optimized set of start values.

In this situation it is of course assumed that all the method steps, calculations, and measurements described, with the exception of the choice of the organ being examined, are carried out with the aid of at least one control facility.

In at least one embodiment, the PET start image data record can be determined from movement-corrected measurement data. As already described at the beginning, the principle is known of subjecting the measurement or raw data to a PET measurement of a movement correction, in order to minimize movement artifacts in the form of smearing. This method can be applied as a supplement to the method according to an embodiment of the invention, in order to reduce movement artifacts. To particular advantage, the at least one transformation requirement can be put to double use: first it is drawn on in order to subject PET measured signals to a movement correction. It is then applied to the PET start image data record, in order to generate a PET image data record.

The PET measurements and the measurements with the imaging facility which is imaging the anatomical features can in this situation be carried out in parallel. Both with computed tomography devices as well as with magnetic resonance facilities, the principle is known of integrating the PET facility, in particular the detector ring. With such hybrid devices, the registration of the image data records can advantageously be avoided.

The recording of the first PET start image data record lasts for a specific period of time, depending on the dose and type of the radiopharmaceutical used and the signal-to-noise ratio required, for example one minute. The PET measurement data measured after the recording of the first PET start image data record can be used either for the generation of further PET start image data records or continuously added to the first PET start image data record in order to increase the signal-to-noise ratio.

Particularly advantageously, the examination object can be moved in a partially controlled manner before the recording of the second anatomy image data record and a second PET start image data record. This embodiment can be used in the assessment of tumors. In this situation the principle is exploited that a tumor which is not visible in the anatomical image data record initiates changes in anatomical image data records if it has grown with the surrounding tissues, e.g. the pleura or the peritoneum, and is moved, since it then causes the surrounding tissue to be moved with it. After the calculation of the transformation requirement(s), these are applied to the PET start image data record.

As a result of this, the movement imaged in the second anatomical image data record in comparison with the first is transferred to the first PET start image data record. If this calculated PET image data record is compared with a PET image data record recorded after the movement, in particular a displacement, of the examination object, then a measurement is obtained for the growth of the tumor and surrounding tissue. If the growth of the tumor is complete, a hundred percent concordance of the calculated and measured PET image data record will be obtained, while if there is no growth, no congruent signals will be derived any longer. These extremes cannot in most cases be achieved in reality, since, for example, even with a fully exposed tumor, the freedom for displacement may be limited due to the adjacent tissue, and therefore concordances are unavoidable.

A displacement of a tumor can be carried out with a needle or a catheter. These interventional instruments are visible in the anatomy image data records. If it is intended that they should also be represented in the PET image data records, then there must be a radiopharmaceutical in them. This can be accommodated in a cavity in the distal end of the interventional instrument, or can even be introduced into the interventional instrument through a hole in the instrument, as late as during an interventional procedure.

In addition to this, the invention also relates to an imaging facility for generating at least one PET or SPECT image data record, comprising an imaging facility which images anatomical features, in particular a magnetic resonance or an X-ray or a computed tomography or an ultrasound facility, a PET facility or a SPECT facility, and a control facility.

The implementation of the foregoing methods in the control facility can in this situation be carried out as software or also as (hard-wired) hardware.

The figures shown hereinafter show the method according an embodiment of to the invention in a simplified manner. On the one hand, the method is represented two-dimensionally, but can equally well be implemented three-dimensionally. For this purpose, the transformation requirements are to be designed as three-dimensional. Moreover, only transformation requirements are shown which are related to translations and the mid-point of the elements. Registration methods are of course known, in particular for elastic registration, and are also possible within the framework of the method according to the invention, which result in more complex transformation requirements for a moved object. Due to these restrictions, the transformation requirements are simplified to two-dimensional vectors, which can be represented by arrows.

The notation with appended Arabic numerals, such as M1, M2, and M3, relates to the representation of the temporal sequence. An MR image data record M2 has therefore been recorded after the MR image data record set M1. If not explicitly mentioned, this should therefore not be interpreted to mean that this is, seen in absolute terms, a first, second, and third MR image data record. The use of appended lower-case letters is analogous to this. These show an indeterminate number of sequences. The notation Mo, however, shows for example that this MR image data record was acquired after the MR image data record Mn.

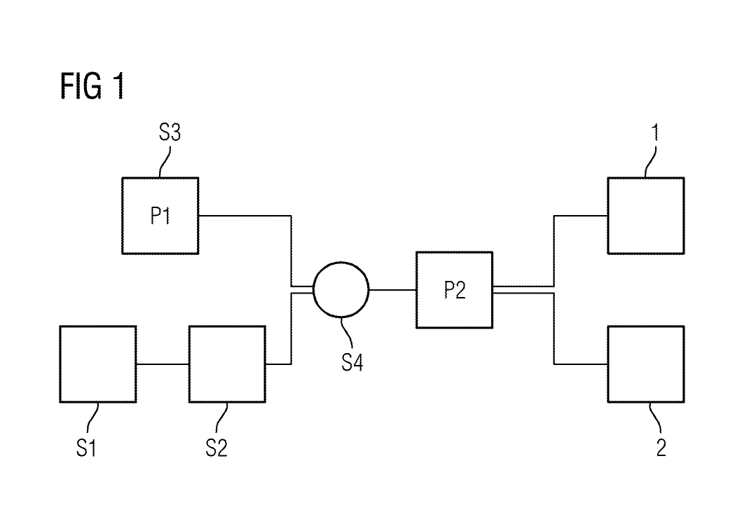

FIG. 1 shows the basic sequence of the method according to an embodiment of the invention. In step S1, two anatomy image data records are recorded, and from these, in step S2 a transformation requirement is determined. Before, during, or after the steps S1 and S2, in step S3 a PET start image data record P1 is recorded. The transformation requirement acquired in step S2 is applied to this PET start image data record P1, as step S4. As a result, the PET image data record P2 is created. This can be displayed in a next step, alone or in combination with the second anatomy image data record, on a display facility 1, and/or stored in a non-volatile memory 2.

The duration of the recording of the two anatomy image data records according to step S1 is a much shorter period, such as two seconds, for example, than the recording of the PET start image data record according to step S3, for example one minute.

As a result, the PET image data record P2 is generated very much faster than if the PET image data record P2 had been measured. In this way it is possible for PET image data records to be updated faster, and, in particular, to be represented in real time. This makes it possible to represent movements with PET image data records which lie below the time resolution of the PET facility.

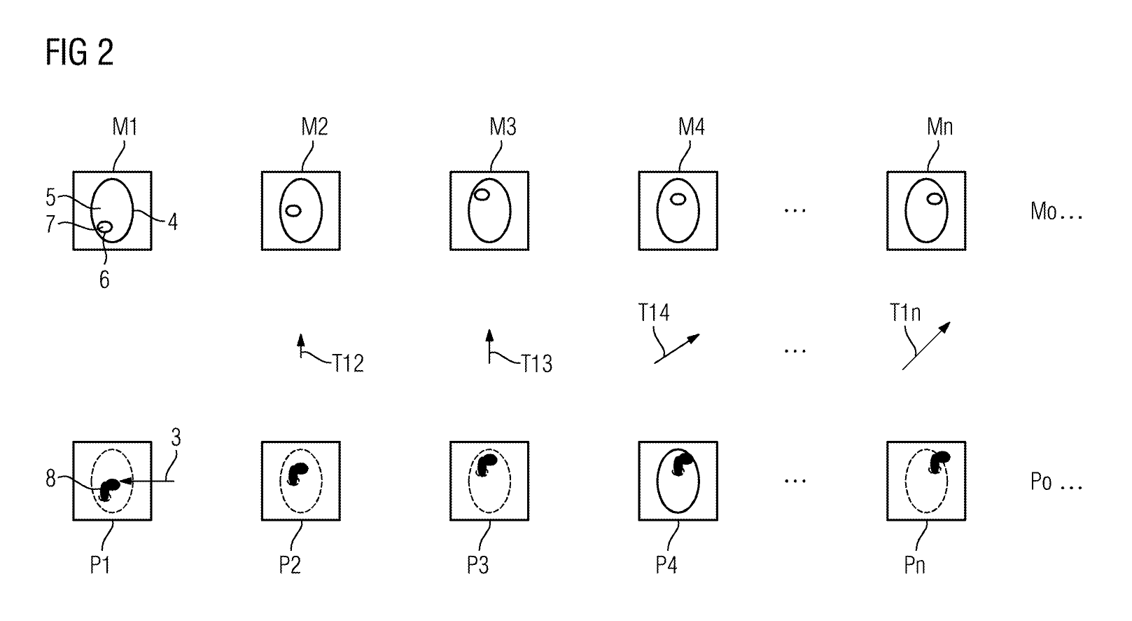

FIG. 2 shows the creation of PET image data records without movement correction, with the aid of a magnetic resonance facility. First, a PET start image data record 2 is recorded and displayed. Because of the movement of the examination object, the signal 3 of the radionuclide imaged in the PET start image data record 2 is smeared. The outline 4 of the examination object is normally not visible, and is only indicated for the purpose of better orientation.

After or during the recording of the PET start image data record P1, as the first anatomy image data record the MR image data record M1 is recorded. This forms a cross-section of the examination object 5 at the level of the stomach 6. The middle of the stomach is symbolized by the mid-point 7. As a recording sequence, a TrueFisp sequence is used, in order to achieve a very short measurement duration.

After the MR image data record M1, the MR image data record M2 is acquired, from the MR image data records M1 and M2 the transformation requirement T12 is determined, and this is applied to the data points of the PET start image data record 2. As has already been described previously, for the simplification of the representation only one transformation requirement T12 is applied onto the mid-point 8 of the PET signal 3. It is of course possible for an individual transformation requirement to be determined for every image element of the PET start image data record P1, in order to take account not only of translational movements but also rotations and extensions. As a result, the PET image data record P2 is obtained, in which the PET-Signal 3 is displaced by the vector contained in the transformation requirement T12.

In the image data records P1, P2, P3, P4 and Pn, for better orientation, the outline of the examination object 5 and of the stomach 6 is represented by broken lines, although these cannot be imaged in the image data records P1, P2, P3, P4 and Pn.

Following on from the MR image data record M2, the MR image data record set M3 is acquired, and, by way of the MR image data records M1 and M3, the transformation requirement T13 is acquired. By the application of T13 onto the PET start image data record P1, the calculated PET image data record P3 is determined.

This sequence continues with the MR image data records M4, . . . , Mn, the transformation requirements calculated in each case T14, . . . , T1n, and the PET image data records P4, . . . , Pn, until a second PET start image data record Po has been measured. The recording of the second PET start image data record Po takes place in parallel to the recording of the MR image data records M2 to Mn.

Because the transformation requirements T12, . . . , T1n were in each case determined starting from the first magnetic resonance image data record M1, this involves complete transformation requirements.

The MR image data record Mo, measured in the end phase of the second PET start image data record Po, forms, with the PET start image data record Po, the new start basis for the determination of new PET image data records Pp, Pq, . . . . In principle, for the performance of the method, the second PET start image data record Po is no longer actually required, but, as a result, the database is kept updated.

While with the pure measurement of PET image data records only the PET start image data records P1 and Po are available with a time interval of approx. one minute, it is possible, via the method according to an embodiment of the invention, for this time interval to be filled with PET image data records P2 to Pn. The PET image data records P1 to Pn therefore form a set of image data which allows for the real-time representation of PET information. As a result, it is possible for this PET information to be used, for example, in interventional procedures.

Within the framework of an examination or of an interventional procedure, MR image data records and PET measurement data can be continuously recorded. After specific time intervals, in each case new PET start image data records will be ready, onto which the newly calculated transformation requirements will be applied.

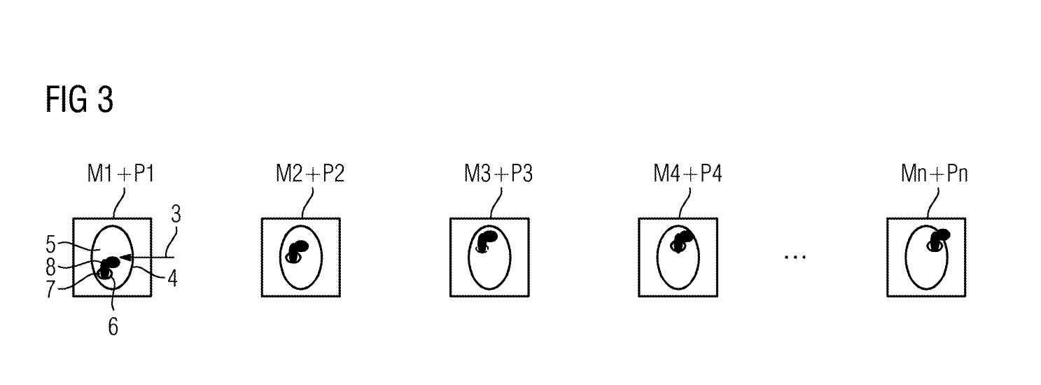

FIG. 3 shows a preferred representation form of the image data records. In this situation, the corresponding MR and PET image data records are shown superimposed. From this is derived a sequence of combination image data records Mi+Pi, wherein i stands for any random number. Every combination image data record is, if required, represented immediately after its calculation on a display facility 1, and in parallel with this is stored in a non-volatile memory 2.

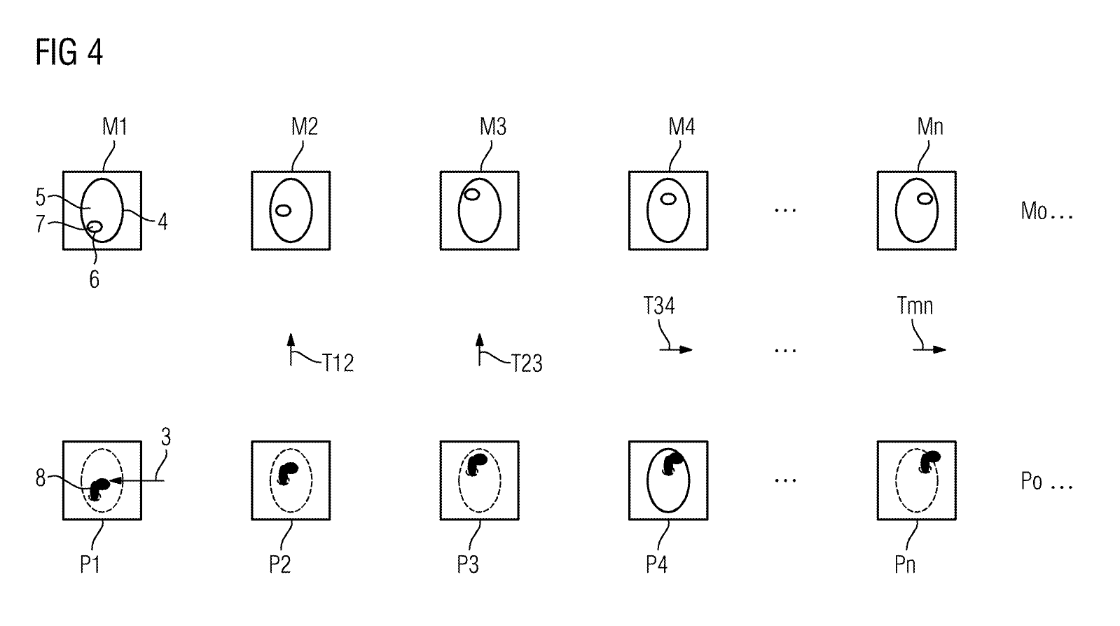

FIG. 4 shows an alternative for the calculation of the transformation requirements. Instead of these always being calculated starting from the first MR image data record M1, they are successively determined from one MR image data record to the next, for example from M3 to M4. As a result, with the corresponding notation, the transformation requirements T12, T23, T34, . . . , Tmn are derived. The transformation requirements are then also no longer all applied onto the PET start image data record P1, but onto the last PET image data record in each case. The application of T23 onto P2 gives P3, T34 onto P3 gives P4, etc. . . . The PET image data record P2 therefore becomes the new PET start image data record.

These transformation requirements are therefore to be applied successively.

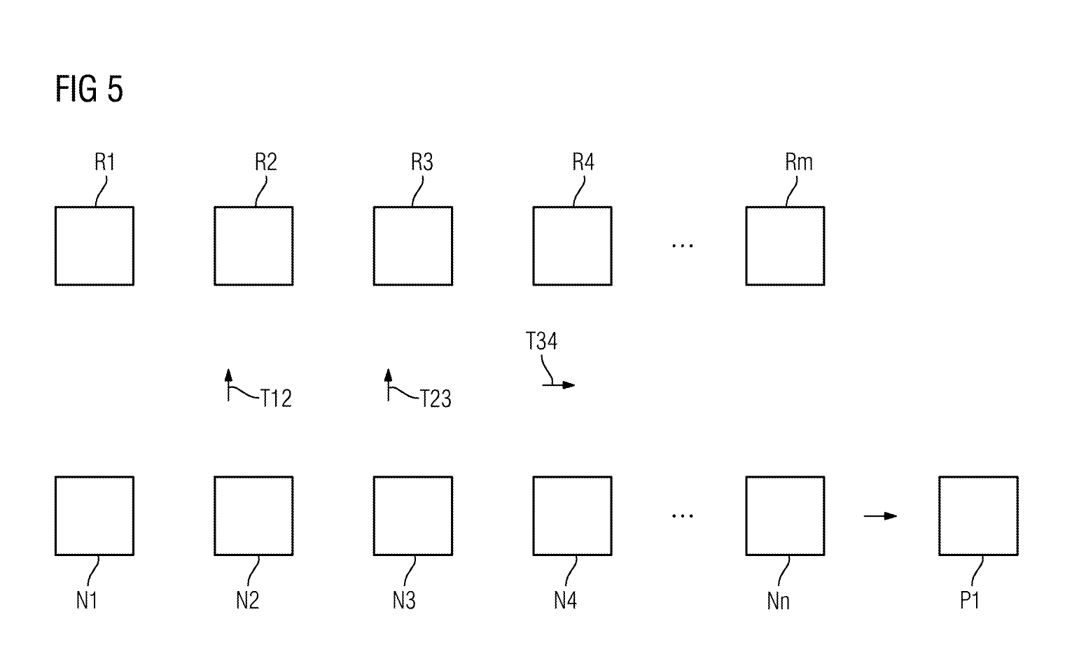

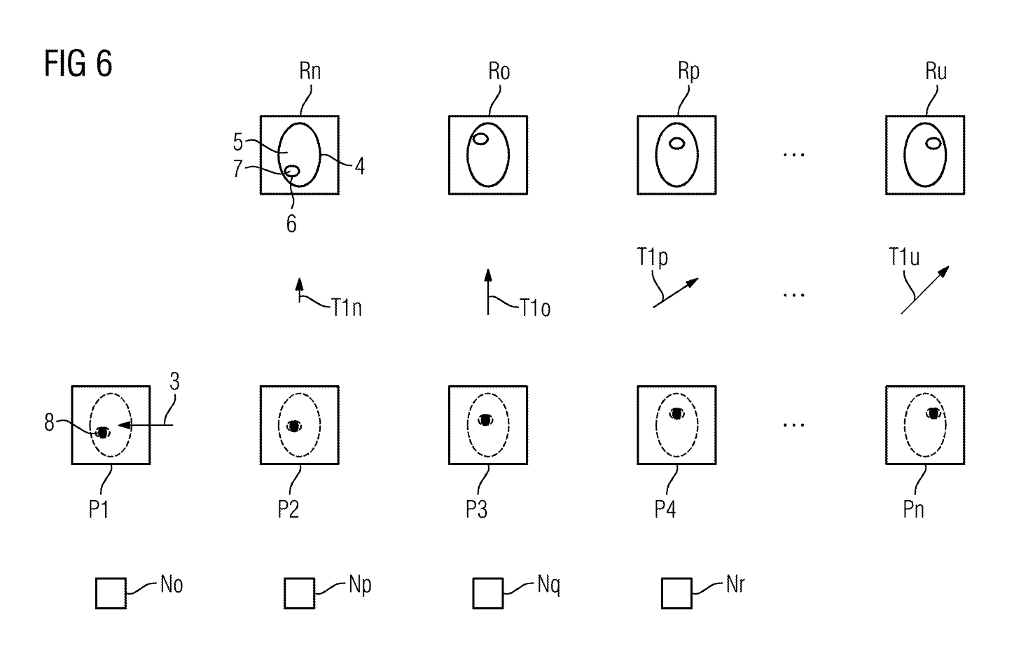

FIGS. 5 and 6 show the method according to an embodiment of the invention with movement-corrected PET image data records. FIG. 5 represents the section up to the calculation of the PET start image data record P1, and FIG. 6 the section which follows. In this situation, for simplification of the representation, the contents of the image, such as the stomach 6, are not shown. In parallel with the measurement data N1, N2, . . . , Nn of the PET facility, in each case X-ray image data records R1, R2, . . . , Rm are acquired. The number of the X-ray image data records R1, R2, . . . , Rm does not necessarily have to correspond to the number of the measurement data elements N1, N2, . . . , Nn. If a measurement data item Ni lies in such a way between the recording of two X-ray image data records that the movement correction can no longer be carried out with the required precision, then an intermediate image data record formed from two X-ray image data records can be interpolated, which corresponds to the movement status of the examination object 5 at the point of time of the recording of the measured data item Ni, in order to gain a corresponding transformation requirement. Specifically, the transformation requirements already described are suitable namely also for the correction of movement artifacts. To carry out the movement correction of the measurement data N1, N2, . . . , Nn, a temporal registration of the PET measurement data N1, N2, . . . , Nn and the X-ray image data records R1, R2, . . . , Rm is accordingly necessary.

After the generation of a first PET start image data record P1 from the movement-corrected measurement data N1, N2, . . . , Nn, the subsequently recorded X-ray image data records Rn, Ro, Rp, . . . are used in order to determine complete transformation requirements T1n, T1o, . . . , and therefore to determine PET image data records P2, P3, . . . . The application of T1n to P1 gives P2, that of T1o to P1 gives P3, etc.

The measurement data No, Np, Nq, . . . of the PET facility further acquired in parallel with the X-ray image data records are movement-corrected with the aid of the X-ray image data records Rn, Ro, Rp, . . . , and added to the PET start image data record P1. This means that the PET start image data record P1 changes continuously after the first creation. In this situation, in particular, the signal-to-noise ratio is improved.

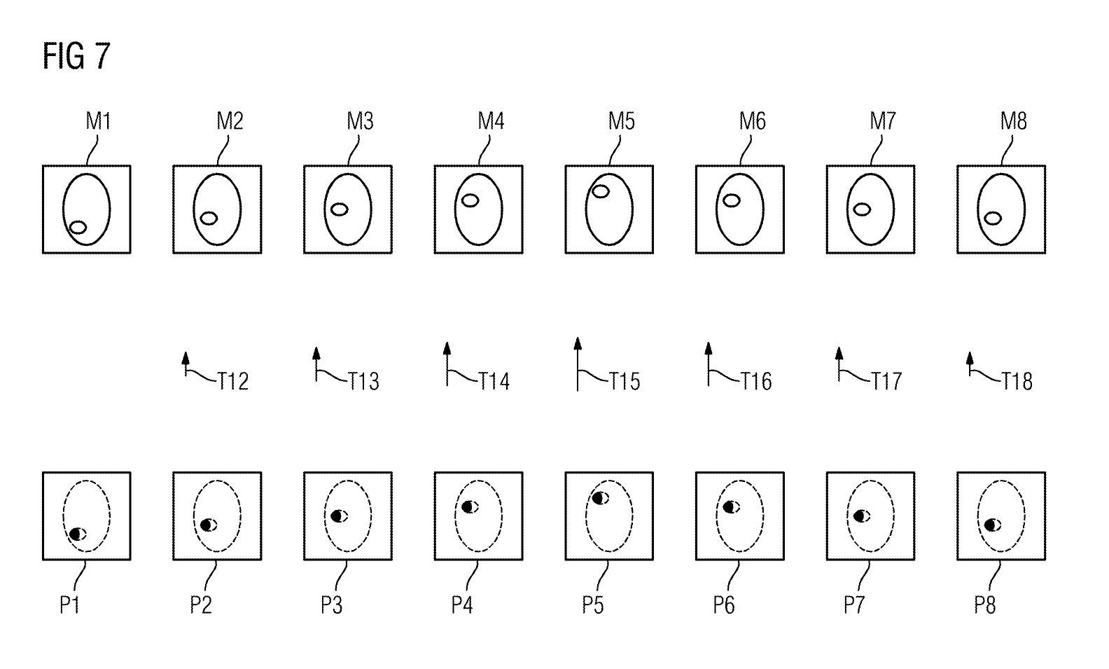

Shown in FIGS. 2, 4, 5 and 6 is the recording of actual real-time image data records, namely the MR image data records M1, . . . , Mo, and the X-ray image data records R1, R2, . . . , Ru. By contrast, FIG. 7 represents the use of pseudo-real-time image data records. The MR image data records M1, . . . , M8 are, for example, recorded directly before or at the beginning of an interventional procedure. The PET start image data record P1 is acquired simultaneously or afterwards.

In order to be able to carry out a movement correction of the PET measurement data, these, like the MR measurements, are triggered onto the Z-wave, or, respectively, the corresponding heart phase is stored as a measurement data item. The MR image data records M1, . . . , M8 image the heart of an examination object in a beat cycle consisting of systole and diastole. The first four MR image data records M1, M2, M3 and M4 image the systole, and the next four MR image data records M5, M6, M7 and M8 image the diastole. From the MR image data records M1, . . . , M8, the successive or the complete transformation requirements are calculated as already described. After the movement correction has been carried out, the PET start image data record P1 are calculated.

From this, making use of the transformation requirements T12, T13, T14, . . . T18 as represented, or alternatively T12, T23, T34, . . . T78, the PET image data records P2, P3, P4, . . . , P8 are calculated. During an interventional procedure, the MR image data records M1, . . . , M8 and the PET image data records P1, . . . , P8 can be combined and, with the use of an EKG, can be displayed at the correct phase of the heart cycle in each case.

How many MR image data records can be recorded in one heart cycle depends, among other factors, on the recording sequence used. The number of 8 MR image data records is purely exemplary.

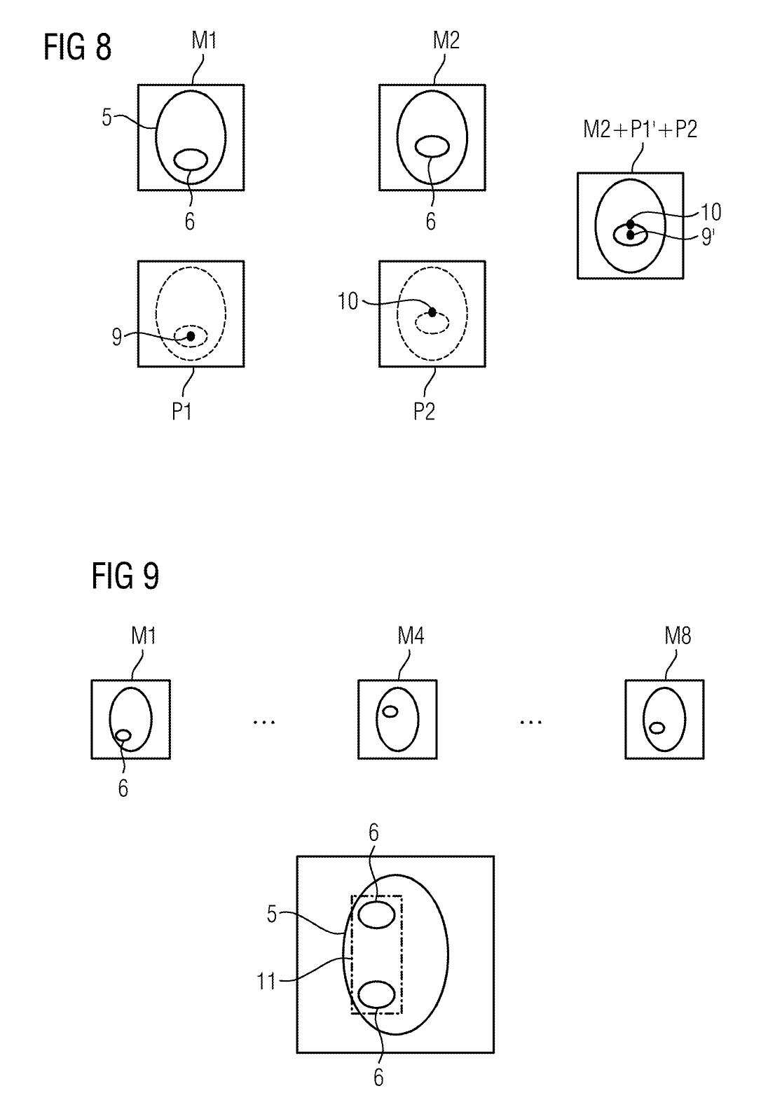

FIG. 8 shows a further embodiment of the method according to an embodiment of the invention. In this situation, within the framework of an interventional procedure, an instrument, for example a needle, is introduced into the examination object, and guided to the examination point, a tumor.

After the recording of the PET start image data record P1 with the measured PET signal 9 and a first MR image data record M1, the needle is guided to the tumor in order to move it in a controlled manner. The recording of the PET measurement data for the recording of a second PET image data record P2, consisting of the PET signal 10 and a further MR image data record MS is then continued.

From the MR image data records M1 and M2, a set of transformation requirements is determined and applied to the PET image data record P1. The use of these transformation requirements leads to the calculated PET signal 9′, and the corresponding PET image data record is designated by P1′. Because the tumor, or, more generally, the examination object in the moved region is not only simply displaced, one single transformation requirement is not sufficient to describe the movement, and therefore a set of transformation requirements is used.

The PET image data record P1′ calculated in this way is compared with the measured PET image data record P2. If the image data or, respectively, the represented PET signals 9′ and 10, deviate from one another, as can be seen on the far right in FIG. 8, the tumor is freely movable, since the movement measured in the PET image data record P2 is not reflected in the MR image data record M2, and therefore was not transferred to the calculated PET image data record P1′. The MR image data record M2 in many cases images only the surrounding tissue, but not the tumor, while with the PET image data record P2 the situation is reversed. A movement which is to be seen in the PET image data record P2 therefore indicates that the tumor and the adjacent tissue have not grown.

Conversely, a concordance of the PET image data records P1′ and P2 or, respectively, of the corresponding signals 9′ and 10, shows that the tumor has grown with the adjacent tissue.

As well as an MR facility, use can of course be made of an X-ray facility, a computed tomography facility, or an ultrasound facility. In order that, with the X-ray facility or the computed tomography facility, the anatomy is imaged and, in particular, the soft tissue around an examination region observed by way of PET, a contrast medium is to be injected if appropriate. All imaging modalities can be used which exhibit a higher time resolution than PET facilities and generate a measurement signal which allows for the calculation of transformation requirements.

FIG. 9 shows the restriction of the determination of the transformation requirements to a part section 11 of the anatomy image data records. The MR image data records M1-M8 cover an entire movement cycle. The MR image data records M2-M8 are elastically registered within the framework of the elastic registration in each case with the MR image data record M1. In this situation a largest and a smallest extension and/or rotation and/or translation of the moved section of the examination object are determined, and in this way a part region 11 of the examination object 5 is determined in which any movements at all take place. On the basis of minor movements of the examination object 5 as a whole, a threshold value can also be specified, which must be exceeded. The determination of the at least one transformation requirement, after the determination of the moved part region 11 of the examination object 5, is restricted to this part region 11. As a result, the calculation of the transformation requirement is accelerated.

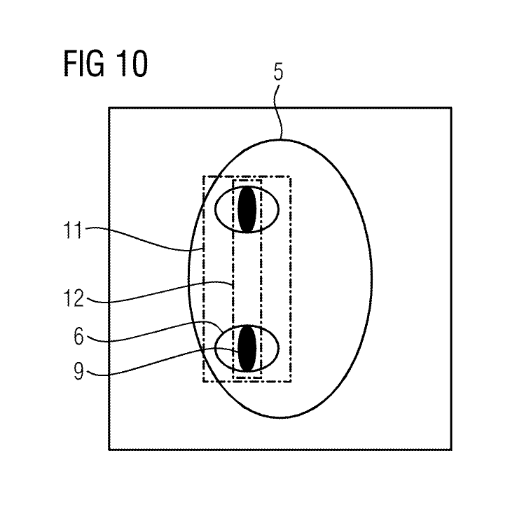

A further acceleration is possible by way of the process represented in FIG. 10. The moved region 12 to be examined of the examination object 5 is further reduced in size due to the fact that transformation requirements are determined only for those image elements of the MR image data record M1 corresponding to the PET start image data record P1, or, more generally, first anatomy image data records, for which, in the PET start image data record P1, a signal 9, in particular a signal above a threshold value, is present. The moved part region 11 in the MR image data record M1 is therefore not completely drawn on, but only the section 12, for which a signal in the PET start image data record P1 is available.

The alternative embodiments:

successive or complete transformation requirements;

actual or pseudo-real-time imaging;

transformation requirements from the entire examination object or from a part region; and/or

with/without movement correction,

can be mixed with one another as desired.

There are, however, partly synergistic effects. For example, the determination of the transformation requirements can be restricted by the undertaking of a movement correction, and thereby by avoiding smearing, to an extremely small image region or part region 11 of the examination object.

What is claimed is:

1. A method for the generation of a PET image data record of an at least partially moved examination object, comprising:
recording or calculating at least one first PET start image data record or one first SPECT start image data record;
recording at least one first anatomy image data record with an imaging facility imaging anatomical features;
recording at least one second anatomy image data record with the imaging facility imaging the anatomical features;
determining at least one transformation requirement from the first and second anatomy image data record; and
generating at least one PET image data record or one SPECT image data record by the application of the at least one transformation requirement onto the PET start image data record or the SPECT start image data record, respectively.

2. The method of claim 1, wherein, as the imaging facility imaging anatomical features, a magnetic resonance facility or a computed tomography facility or an X-ray facility or an ultrasound facility is used.

3. The method of claim 1, wherein the at least one transformation requirement is carried out by an elastic registration of the first anatomy image data record with the second anatomy image data record.

4. The method of claim 1, wherein, a part region of respective ones of the first and second anatomy image data record is used for the determination of the at least one transformation requirement.

5. The method of claim 1, wherein the part region from at least one of the first and second anatomy image data record is determined automatically.

6. The method of claim 1, wherein the PET start image data record or the SPECT start image data record is determined from movement-corrected measurement data.

7. The method of claim 1, wherein the PET measurement data recorded after the recording of the PET start image data record is added to the PET measurement data used for the generation of the PET start image data record, in order to obtain a PET start image data record with an improved signal-to-noise ratio.

8. The method of claim 1, wherein the PET measurement data recorded after the recording of the first PET image data record is used to create at least one further PET start image data record.

9. The method of claim 1, wherein the recording at least one second anatomy image data record, the determining and the generating are carried out several times.

10. An imaging facility for creating at least one PET image data record or one SPECT image data record, comprising:
an imaging facility configured to image anatomical features; and
a control facility, configured to at least
record or calculate at least one first PET start image data record or one first SPECT start image data record,
record at least one first anatomy image data record with an imaging facility imaging anatomical features,
record at least one second anatomy image data record with the imaging facility imaging the anatomical features,
determine at least one transformation requirement from the first and second anatomy image data record, and
generate at least one PET image data record or one SPECT image data record by the application of the at least one transformation requirement onto the PET start image data record or the SPECT start image data record, respectively.

11. The method of claim 4, wherein the part region from at least one of the first and second anatomy image data record is determined automatically.

12. The method of claim 3, wherein, a part region of respective ones of the first and second anatomy image data record is used for the determination of the at least one transformation requirement.

13. The method of claim 12, wherein the part region from at least one of the first and second anatomy image data record is determined automatically.

14. The imaging facility of claim 10, wherein the imaging facility is a magnetic resonance facility or an X-ray facility or a computed tomography facility or an ultrasound facility, a
PET facility or an SPECT facility.
